(19) United States
(12) Patent Application Publication
(10) Pub. No.: US 2011/0027852 A1
(43) Pub. Date: Feb. 3, 2011

(54) Processing lignocellulosic biomass to fixed, high levels of dry matter content.

(76) Inventor: Jan Larsen, Tommerup (DK)

Correspondence Address: Plougmann & Vingtoft A/S, Sundkrogsgade 9, PO Box 831, Copenhagen, 2100, DK

(21) Appl. No.: 12/935,587

(22) PCT Filed: Apr. 14, 2009
(86) PCT No.: PCT/IB09/05231

§ 371 Date: Sep. 30, 2010

Related U.S. Application Data
(60) Provisional application No. 61/043,743, filed on Apr. 10, 2008.

(30) Foreign Application Priority Data
Apr. 10, 2008 (DK) PA 2008 00524

(51) Int. Cl. C12P 1/02

(52) U.S. Cl. 435/171

(57) ABSTRACT

The invention relates in general to methods of processing lignocellulosic biomass and to methods of pre-treatment of lignocellulosic pretreated with steam biomass. In particular, the invention provides methods which fix moisture levels in lignocellulosic biomass to levels near the inherent water holding capacity of the material.

### FIELD OF THE INVENTION

The invention relates in general to methods of processing lignocellulosic biomass and to methods of pre-treatment of lignocellulosic biomass. In particular, the invention provides methods which fix moisture levels in lignocellulosic biomass to levels near the inherent water holding capacity of the material.

### BACKGROUND

Bioethanol offers a promising alternative to fossil fuels, providing renewable and “carbon neutral” energy sources that do not disrupt global atmospheric carbon dioxide balance. Amongst other possible sources of bioethanol precursors, lignocellulosic biomass can be enzymatically hydrolysed to provide fermentable carbohydrates. However, because of its complex chemical structure, lignocellulose can only be efficiently hydrolysed by presently known enzyme activities after some pre-treatment that renders cellulose fibers accessible to enzyme catalysis. Such pre-treatment processes typically involve heating to comparatively high temperatures, between 100 and 250° C. Large scale bioethanol production from lignocellulosic biomass requires large scale pre-treatment and processing. Accordingly, an intense interest has arisen in methods of biomass pre-treatment and processing that reduce costs or otherwise increase commercial viability of bioethanol on production scale.

Two factors which heavily influence overall costs of bioethanol production from lignocellulosic biomass are energy costs of ethanol distillation from fermentation mixtures and energy costs of biomass pre-treatment.

Energy costs of ethanol distillation can be greatly reduced where ethanol content of fermentation mixtures exceeds 4%. However, to achieve these high ethanol levels in fermentation mixtures, without requiring costly and inefficient additional process steps, enzymatic hydrolysis of pre-treated lignocellulosic biomass should be conducted at relatively high dry matter content (DM)—at least about 15-20%. Previous attempts to achieve high DM content in fermentation mixtures have been hampered by accumulation of fermentation inhibitors generated during pre-treatment and by other problems arising during enzyme hydrolysis and fermentation. See e.g. refs. 1-7.

Recently, however, production scale methods for enzyme hydrolysis of pre-treated lignocellulosic biomass have been reported that are efficient and effective at DM greater than 20%. These methods provide liquefaction and saccharification of biomass using “free fall” mixing, as described by WO 2006/56838 (ref. 8), which is hereby incorporated by reference in entirety.

Energy costs of pre-treatment can be reduced where biomass is pre-treated at high DM. Greater dry matter content of biomass corresponds with reduced aqueous content. Thus, the greater the dry matter content of biomass during pre-treatment, the less energy is wasted heating aqueous content. It is, thus, generally advantageous during pre-treatment to achieve the highest possible DM levels (lowest possible aqueous levels) of lignocellulosic biomass that do not contribute to eventual reduction of ethanol yield (% theoretical) from fermentation mixtures.

Optimal pre-treatment conditions require that biomass have some aqueous content. Eventual ethanol yield (% theoretical) from lignocellulosic biomass is generally improved to the extent that it is pre-treated under conditions in which cellulose fibers do not contain air. Biomass that is simply exposed to moisture can, eventually, with time, achieve homogenous aqueous saturation of cellulose fibers. However, such an “impregnation” approach is slow, and accordingly unsuitable for production scale pre-treatment. Aqueous content of biomass has been previously optimized on production scale by soaking and pressing prior to pre-treatment, for example, as described by WO 2007/009463 (ref. 9), which is hereby incorporated by reference in entirety. After soaking in an excess of aqueous solution, then pressing to remove as much aqueous content as possible, lignocellulosic biomass will typically comprise a “saturation level” of aqueous content corresponding to DM greater than about 30%.

While such soaking and pressing methods are effective, they require additional energy for pressing, time delays for soaking, as well as additional process steps. These introduce additional costs and production inefficiencies.

Accordingly, it is advantageous to provide methods of processing lignocellulosic biomass, suitable for use in continuous processing on production scale, that provide homogenous, aqueous saturation of cellulose fibers quickly, with low energy cost, and with the fewest possible process steps.

### SUMMARY OF THE INVENTION

In some embodiments, the invention provides methods of processing lignocellulosic biomass whereby biomass is wetted with an amount of aqueous solution sufficient to provide moisture levels near the inherent water holding capacity of the material then thoroughly mixed, optionally using a mixer that massages water content into lignocellulosic fibers.

### BRIEF DESCRIPTION OF THE DRAWINGS

FIG. 1 shows a mixer used in preferred embodiments to massage water content in lignocellulose fibers.

FIG. 2 shows alternative arrangements of water or aqueous solution addition and a mixer suitable for practice of embodiments of the invention in a continuous pre-treatment process.

FIG. 3 shows cellulose conversion (%) as a function of time of enzymatic hydrolysis of lignocellulosic biomass pre-treated by methods of the invention at fixed dry matter content from 20 to 50%.

FIG. 4 shows the effect of mixing time (from 10-30 minutes) on cellulose conversion (%) as a function of time of enzymatic hydrolysis of lignocellulosic biomass processed by methods of the invention to fixed dry matter content of 35%.

### DETAILED DESCRIPTION OF PREFERRED EMBODIMENTS

As used herein, the following terms have the following meanings:

### (i). Lignocellulosic Biomass

Lignocellulosic biomass refers to material derived from plants or other organisms in which carbohydrate content is substantially cellulose and hemicellulose and which comprises more than 5% lignin. Cellulose is a polysaccharide composed of D-glucose monomers linked by β-1,4-glucosidic bonds with a degree of polymerisation up to 10,000. Hemicellulose is a complex heterogeneous polysaccharide comprising different monomer residues including : D-glucose, D-galactose, D-mannose, D-xylose, L-arabinose, D-glucuronic acid and 4-O-methyl-D-glucuronic acid having a degree of polymerisation below 200. Lignin is a complex aromatic network formed by polymerisation of phenyl propane and comprising monomers including: ρ-coumaryl alcohol, coniferyl alcohol and sinapyl alcohol, typically linked through arylglyceryl-β-aryl ether bonds. The term as used herein includes processed materials, such as papers, as well as primarily natural materials, such as agricultural wastes. Lignocellulosic biomass will typically comprise water content. A mixture of water and/or other agents and/or solvents comprising lignocellulosic biomass as the predominant solid component can also be referred to as “a” lignocellulosic biomass within the meaning of the term as used. The carbohydrate composition of a lignocellulosic biomass may be changed during pre-treatment.

### (ii). Dry Matter

Dry matter refers to insoluble material. Typically, dry matter comprises insoluble fibers.

(iii). Inherent Water Holding Capacity of the Biomass.

Inherent water holding capacity of the biomass refers to the amount of water, or aqueous solution, that remains after repeated “pressing” in a biomass that has been “soaked” in a “soaking and pressing” process such as that described in WO 2007/009463.

### (iv). Fixed Dry Matter Content and Thorough Mixing

Fixed dry matter content refers to moisture content of lignocellulosic biomass adjusted prior to pre-treatment and/or enzymatic hydrolysis. The dry matter content is adjusted or “fixed” by adding a quantity of water, or aqueous solution comprising one or more chemical additives, sufficient to provide moisture levels between 80-120% of the inherent water holding capacity of the biomass then thoroughly mixing. Mixing is “thorough” where substantially all of the dry matter of the lignocellulosic biomass is wetted by added water or aqueous solution. Dry matter content is “fixed” where substantially all of the water, or aqueous solution, is incorporated within fibers with substantially no excess water, or aqueous solution, that is not incorporated within fibers, except an amount not exceeding an amount of water, or aqueous solution, added in excess of 100% of the inherent water holding capacity of the biomass. Soaking typically involves excess water, >120% of the inherent water holding capacity of the biomass, that is not incorporated within fibers and does not provide fixed dry matter content as used herein.

### (v). Massages Water Content

Water content is massaged into wetted biomass fibers by subjecting them to a form of mixing that acts to alternately compress fibers then restore them to a relaxed state. An example of a mixer that massages water content into wetted biomass fibers is the Cormall Multimix MTX two auger livestock feed mixer.

### (vi). Pre-Treatment

Pre-treatment refers to a manipulation of lignocellulosic biomass that renders its cellulosic component more accessible to enzymes that convert carbohydrate polymers into fermentable sugars. Heat pre-treatment refers to a pre-treatment in which biomass is heated to temperatures of 100° C. or more.

(vii). Enzymatic Hydrolysis

Enzymatic hydrolysis refers to treatment of a lignocellulosic biomass with a mixture of enzyme activities comprising one or more cellulytic enzyme in such manner as to convert cellulose content to carbohydrates with at least 20% theoretical yield.

Some embodiments provide a method of processing lignocellulosic biomass comprising;
providing a lignocellulosic biomass
adding an amount of water or aqueous solution sufficient to provide moisture levels between 80-120% of the inherent water holding capacity of the biomass, and
mixing in a mixer that imparts a combination of shear and/or pressing forces such that the biomass is mixed thoroughly within 60 minutes, or, optionally, within 30 minutes, or, optionally, within 20 minutes, or, optionally within 10 minutes wherein said mixed biomass having fixed dry matter content is subsequently subject to heat pre-treatment and/or enzymatic hydrolysis.

Other embodiments provide a method of processing lignocellulosic biomass comprising;
providing a lignocellulosic biomass
an amount of water or aqueous solution sufficient to provide moisture levels between 80-120% of the inherent water holding capacity of the biomass, and
mixing thoroughly in a mixer that massages water content into lignocellulosic fibers wherein said mixed biomass having fixed dry matter content is subsequently subject to heat pre-treatment and/or enzymatic hydrolysis.

Embodiments of the invention may be practiced in batch, semi-continuous or continuous modes of operation.

In preferred embodiments, the dry matter content is fixed to levels corresponding to moisture content of greater than 85% but less than 100% of the inherent water holding capacity of the biomass. In more preferred embodiments, the dry matter content is fixed to levels corresponding to moisture content of greater than 95% but less than 100% of the inherent water holding capacity of the biomass. In still more preferred embodiments, other embodiments, the dry matter content is fixed to levels corresponding to moisture content about 100% of the inherent water holding capacity of the biomass.

In preferred embodiments, dry matter content of lignocellulosic biomass is fixed on a large scale, having dry matter mass at least 40 kg, or having dry matter mass greater than 50 kg, or greater than 100 kg, or greater than 1000 kg, or greater than 10,000 kg.

In the practice of some embodiments, any suitable lignocellulosic biomass feedstock having intrinsic dry matter content greater than about 50% may be used including at least corn stover, wheat straw, rice straw, bagasse, corn fiber, hardwood bulk, softwood bulk, nut shells, corn cobs, grasses, including but not limited to coastal Bermuda grass and switch grass, paper, including newspaper, waste papers and paper from chemical pulps, sorted refuse, cotton seed hairs, empty fruit baskets and other materials well known in the art.

The lignocellulosic biomass may be pre-processed by chopping, grinding, ball milling, or other mechanical treatment processes.

In preferred embodiments, a lignocellulosic biomass will have a distribution of particle sizes prior to pre-treatment having 80% falling within the range of 1 to 10 cm. In other embodiments, a lignocellulosic biomass will have a distribution of particle sizes having 80% falling within the range of 0.5 to 15 cm.

In practice of preferred embodiments, it is helpful to determine the inherent water holding capacity of a lignocellulosic biomass, for example, by measuring the moisture content that remains after “pressing” in a biomass that has been “soaked” in a “soaking and pressing” process such as that described in WO 2007/009463. For example, wheat straw typically has an inherent water holding capacity corresponding to about 42% DM.

In preferred embodiments, intrinsic DM content of a lignocellulosic biomass is first determined by means of drying to no loss of weight or by any method known in the art. A quantity of water, or aqueous solution, sufficient to provide moisture levels between 80-120% of the inherent water holding capacity of the biomass can then be readily determined based on the dry mass of the lignocellulosic biomass. For example, for 10,000 kg of wheat straw having dry matter content 92.0%, 30,000 liters of water or aqueous solution should be added to provide dry matter content of about 30% (moisture content about 120% of the inherent water holding capacity). For the same lignocellulosic biomass, to provide dry matter content of about 40% (moisture content about 103% of the inherent water holding capacity), only 23,000 liters of water or aqueous solution need be added.

In other embodiments, dry matter content of a lignocellulosic biomass can be estimated visually, or based upon reference materials or prior experience.

In other embodiments, an appropriate amount of water or aqueous solution can be approximated or added in amounts that may vary within constraints of some process limitations such as water availability. For example, dry matter content may be fixed imprecisely at between 30-40% by adding an amount of water or aqueous solution that is not precisely measured, although sufficient, in that it does not exceed the amount required for 30% dry matter.

Aqueous solutions suitable for practice of some embodiments may comprise acids, bases, salts, metals, or other chemical additives, enzymes or microorganisms. In preferred embodiments, a mildly acidic solution is added. Optimum pH is typically between 3.5-4.0. This lowers heat requirements for pre-treatment and prevents sticking of “cooked” biomass to reactor vessels or communication lines. Wash effluent or extracts of pre-treated biomass, typically containing acetic acid, may be added as aqueous solutions suitable for practice of some embodiments.

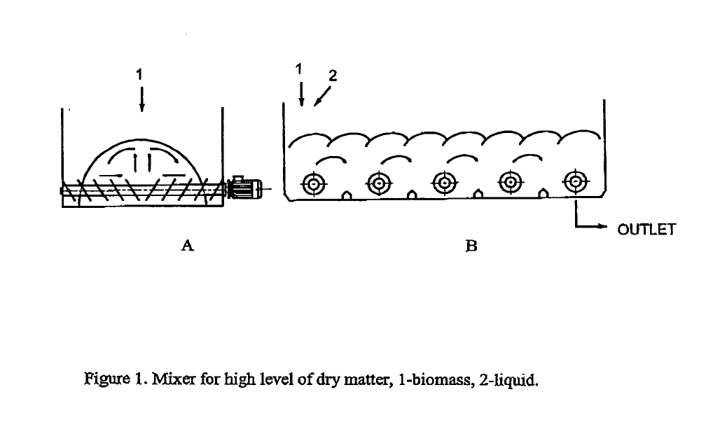

In preferred embodiments, the lignocellulosic biomass and added water or aqueous solution may be mixed thoroughly using a mixer that massages water content into lignocellulosic fibers. One such mixer, suitable for practice of preferred embodiments, is illustrated in FIG. 1. The mixer is mounted with a series of augers, in this example 5. FIG. 1 (A) provides an end view while FIG. 1 (B) provides a side view of a preferred mixer. As shown in FIG. 1 (B), the mixer comprises a series of augers, in this example 5, mounted perpendicular to the flow of biomass. As shown in FIG. 1 (A), each auger has blades that are situated to provide, along the same axis, counterposing helical motion from each end, such that biomass from either end is transported towards the center of the auger. At the center of the auger, biomass is “shot” up, in what can be described as a “molehill” of water. Added biomass is “shot” up then naturally falls back down into the augers. Some of the biomass from each “molehill” moves forward through the mixer to the next auger in series. A steady state flow of biomass into and out of the mixer is established by constant flow of biomass into one end of the mixer and constant removal of thoroughly mixed biomass from an outlet situated at the opposite end of the mixer. The time of mixing can be adjusted by adjusting the rate of removal of thoroughly mixed biomass, and thus the rate of introduction of unmixed biomass. Other suitable mixers that massage water content into lignocellulosic fibers include at least mixers similar to any of the mixers described in WO8002458, US20070274151, WO07089144, WO07083998, US20050105390, US20050094486, and US20030169639 (refs. 10-16).

In other embodiments, the lignocellulosic biomass and added water or aqueous solution may be mixed thoroughly using a mixer that imparts a combination of shear and/or pressing forces such that the biomass is mixed thoroughly within 60 minutes, or, optionally, within 30 minutes, or, optionally, within 20 minutes, or, optionally, within 10 minutes. In still other embodiments, the lignocellulosic biomass and added water or aqueous solution may be mixed by any means that provides that, within 60 minutes, or, optionally, within 30 minutes, or, optionally, within 20 minutes, or, optionally, within 10 minutes, substantially all of the water, or aqueous solution, is incorporated within fibers with substantially no excess water, or aqueous solution, that is not incorporated within fibers.

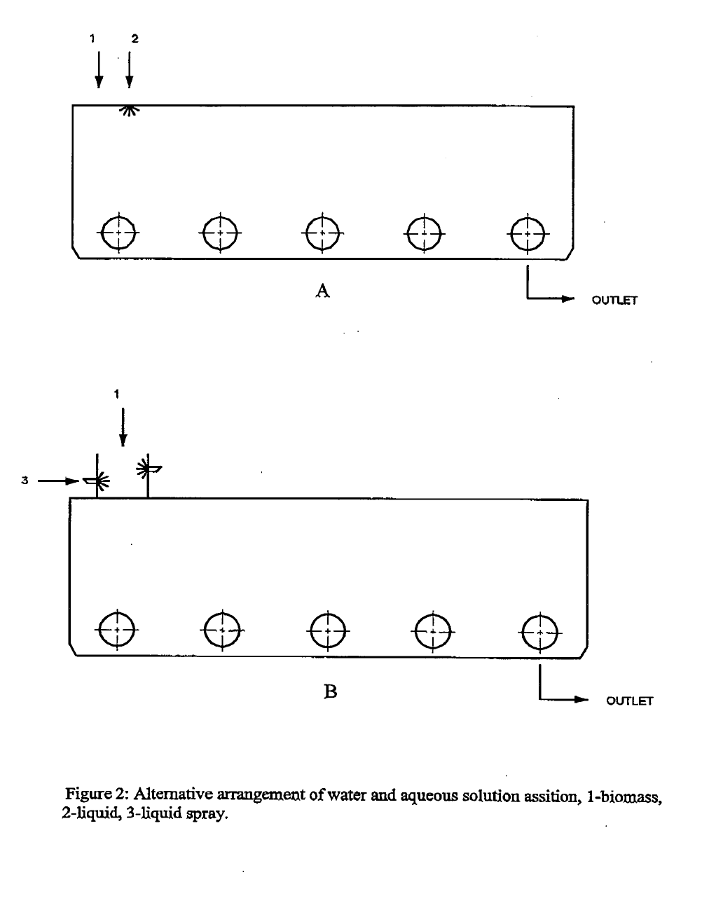

In practice of some embodiments, water or aqueous solution may be added as cold liquid, which is typically absorbed in a shorter time, or as steam or a combination of steam and liquid. In practice of some embodiments, water or aqueous solution may be added directly in the mixer. Alternatively, water or aqueous solution may be added within a vertical column through which biomass is falling, by force or gravity or conveyance, into the mixer. Other possible arrangements can be readily imagined. FIG. 2 illustrates two alternative arrangements of water or aqueous solution addition and a mixer suitable for practice of embodiments of the invention in a continuous pre-treatment process. In the most preferred embodiment, biomass is added to the mixer simultaneously with an appropriate quantity of water or aqueous solution. Alternatively, biomass may be sprayed, for example, as it is falling through a column that transports biomass.

After processing by embodiments of the invention, the biomass can be pre-treated by any heat pre-treatment and, further, to any post pre-treatment processing.

In some embodiments, a biomass that does not require pre-treatment may be used. For example, waste paper and other paper pulp feedstocks, do not require heat pre-treatment but can be used directly in enzymatic hydrolysis.

### Example 1

These data origin from the IBUS pilot plant of Inbicon in Fredericia, Denmark. Cut wheat straw and liquid were mixed in a KUHN Euromix II type 1460 feed mixer. The dry matter content of the wetted straw after mixing was varied from 20 to 50% DM, which corresponds with moisture levels of between 138 to 86% of the inherent water holding capacity of the biomass.

500 kg of cut (2-10 cm in length) wheat straw was added to the mixer. Liquid in an adjusted amount was sprayed on the straw. Then the mixer was started, and the liquid was massaged in to the straw. Residence time in the mixer was 30 minutes. After mixing the dry matter content was measured in the wetted straw and it was found to be in agreement with the calculated. In this way samples of wetted wheat straw with a content of 20, 30, 40 and 50±1% DM were prepared. Two samples were prepared at 40% DM, which corresponds with moisture levels of about 103% of the inherent water holding capacity of the biomass.

These samples were loaded in to the pilot pre-treatment facilities of Inbicon. In this pilot plant the wetted wheat straw in a continuously way was steam treated at 185° C. for 10 minutes.

As a reference, for comparison with the “fixed dry matter” samples, soaked and pressed wheat straw was also pre-treated. In the reference sample,s cut straw was soaked for 5-10 minutes in 80° C. hot liquid. After the soaking the straw was pre-treated at a dry matter content of 18-22%, which corresponds with moisture levels of between 141 to 134% of the inherent water holding capacity of the biomass.

The pre-treatment must ensure that the structure of the lignocellulosic content is rendered accessible for enzymatic hydrolysis, and at the same time the concentrations of harmful inhibitory by-products such as acetic acid, furfural and hydroxymethyl furfural remain substantially low. Therefore, after heat pre-treatment, the pre-treated straw is washed by water or condensate then pressed. After post-pre-treatment washing and pressing, the cellulosic fibres have a dry matter content of app. 25-35%. The pre-treated straw was collected in plastic bags and stored at 1-5° C. until use.

The pre-treated wheat straw samples were evaluated regarding convertibility of cellulose in a shake flask set up at 12% DM, where the samples were simultaneous saccharified and fermented (SSF). The pre-treated fibre fraction was diluted with an acetic acid buffer, pre-hydrolysed 6 hours with Novozym 188 and Celluclast 1.5 FG at 50° C. using an enzyme loading of 5.0 FPU (g DM)⁻¹ then simultaneously saccharified and fermented (SSF) 144 hours at 30-33° C. with common bakers yeast (Baker's yeast, De Danske Spritfabrikker).

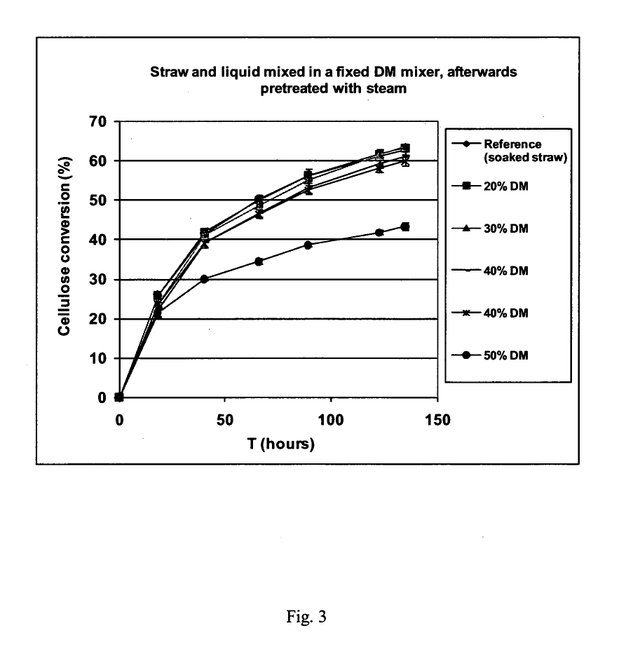

FIG. 3 shows that fixing dry matter of biomass prior to steam pre-treatment at levels of dry matter from 30-40% corresponding to from 120 to 103% of the water holding capacity of the biomass provides equivalent yields in cellulose conversion compared to soaking, typically 18-22% dry matter, corresponding to from 141 to 134% of the inherent water holding capacity.

Accordingly, by practice of embodiments of the invention, it is possible to reduce energy consumption, stream line process steps, and reduce process time without loss of yield, by reducing the water content of the biomass during pre-treatment. However, as shown in FIG. 1, at dry matter content 50%, corresponding to only about 86% of the water holding capacity of the biomass, yields in cellulose conversion are reduced considerably relative to soaking.

### Example 2

These data origin from the IBUS pilot plant of Inbicon in Fredericia, Denmark. Cut wheat straw and liquid were mixed in a KUHN Euromix II type 1460 feed mixer. The dry matter content of the wetted straw after mixing was 35%, which corresponds to about 112% of the water holding capacity of the biomass.

500 kg of cut (2-10 cm in length) wheat straw was added to the mixer. A pre-determined amount of aqueous solution, sufficient to provide dry matter content of about 35%, was sprayed on the straw. Then the mixer was started, and the liquid was massaged in to the straw. Residence time in the mixer was varied from 10 to 30 minutes. After mixing the dry matter content was measured in the wetted straw and it was found to be in agreement with the calculated. In this way samples of wetted wheat straw with a content of 35±1% DM were prepared.

These samples were loaded in to the pilot pre-treatment facilities of Inbicon. In this pilot plant the wetted wheat straw in a continuously way was steam treated at 185° C. for 10 minutes.

The pre-treatment must ensure that the structure of the lignocellulosic content is rendered accessible for enzymatic hydrolysis, and at the same time the concentrations of harmful inhibitory by-products such as acetic acid, furfural and hydroxymethyl furfural remain substantially low. Therefore, after heat pre-treatment, the pre-treated straw is washed by water or condensate then pressed. After post-pre-treatment washing and pressing, the cellulosic fibres have a dry matter content of app. 25-35%. The pre-treated straw was collected in plastic bags and stored at 1-5° C. until use.

The pre-treated wheat straw samples were evaluated regarding convertibility of cellulose in a shake flask set up at 12% DM, where the samples were simultaneous saccharified and fermented (SSF). The pre-treated fibre fraction was diluted with an acetic acid buffer, pre-hydrolysed 6 hours with Novozym 188 and Celluclast 1.5 FG at 50° C. using an enzyme loading of 5 FPU (g DM)⁻¹ then simultaneously saccharified and fermented (SSF) 400 hours at 30-33° C. with common bakers yeast (Baker's yeast, De Danske Spritfabrikker).

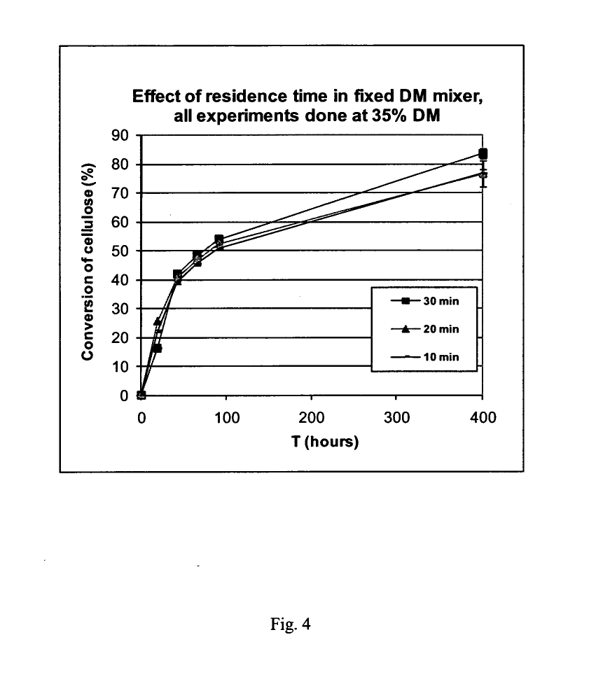

FIG. 4 shows that residence times between 10 and 30 minutes in a fixed dry matter mixer before steam pre-treatment ensures equal yield in cellulose conversion.

Accordingly, by practice of embodiments of the invention, it is possible to reduce energy consumption, stream line process steps, and reduce process time without loss of yield, by reducing the water content of the biomass during pre-treatment, through a processing that provides thorough mixing within 60 minutes, or, optionally, within 30 minutes, or, optionally, within 20 minutes, or, optionally within 10 minutes.

### Example 3

These data origin from the IBUS pilot plant of Inbicon in Fredericia, Denmark. Dried empty fruit bunches (EFB) of oil palm and liquid were mixed in a KUHN EUROMIX II™ type 1460 feed mixer.

500 kg of EFB (average fibre length of app. 5-10 cm) was added to the mixer. Liquid in an adjusted amount was sprayed on the EFB. Then the mixer was started, and the liquid was massaged in to the EFB. Residence time in the mixer was 60 minutes. After mixing the dry matter content was measured in the wetted EFB and it was found to be in agreement with the calculated. In this way samples of wetted EFB with a content of 25 and 35±1% DM were prepared.

These samples were loaded in to the pilot pre-treatment facilities of Inbicon. In this pilot plant the wetted EFB in a continuously way was steam treated at 200° C. for 12 minutes.

The pre-treatment must ensure that the structure of the lignocellulosic content is rendered accessible for enzymatic hydrolysis, and at the same time the concentrations of harmful inhibitory by-products such as acetic acid, furfural and hydroxymethyl furfural remain substantially low. Therefore, after heat pre-treatment, the pre-treated EFB is washed by water or condensate then pressed. After post-pre-treatment washing and pressing, the cellulosic fibres had a dry matter content of app. 25-35%. The pre-treated EFB was collected in plastic bags and stored at 1-5° C. until use.

The pre-treated EFB samples were evaluated regarding convertibility of cellulose in a shake flask set up at 12% DM, where the samples were simultaneously saccharified and fermented (SSF). The pre-treated fibre fraction was diluted with an acetic acid buffer, pre-hydrolysed 6 hours with ACCELLERASE 1500™ (Genencor) at 50° C. using an enzyme loading of 0.21 ml (g cellulose)-1 then simultaneously saccharified and fermented (SSF) 144 hours at 30-33° C. with common bakers yeast (Baker's yeast, De Danske Spritfabrikker). In these experiments a cellulose conversion of 88% was reached.

The examples and descriptions provide representative examples of particular embodiments and are not intended to limit the scope of the invention.

### REFERENCES

1 P. Sassner et al., “Bioethanol production based on simultaneous saccharification and fermentation of steam-pre-treated Salix at high dry matter content,” Enzyme and Microbial Technology (2006) 39:756;
2 M. Alkasrawi et al., “Influence of strain and cultivation procedure on the performance of simultaneous saccharification and fermentation of steam pre-treated spruce,” Enzyme and Microbial Technology (2006) 38:279;
3 A. Rudolf et al., “A comparison between batch and fed-batch simultaneous saccharification and fermentation of steam pre-treated spruce,” Enzyme and Microbial Technology (2005) 37:195;
4 M. Ballesteros et al., “Ethanol production from paper material using a simultaneous saccharification and fermentation system in a fed-batch basis,” World Journal of Microbiology & Biotechnology (2002), 18:559.
5 Charlotte Tengborg, Mats Galbe, and Guido Zacchi: Influence of Enzyme Loading and Physical Parameters on the Enzymatic Hydrolysis of Steam-Pre-treated Softwood Biotechnol. Prog. 2001, 17, 110-117;
6 Hanne R. Sørensen,†,‡ Sven Pedersen,† and Anne S. Meyer*,‡: Optimization of Reaction Conditions for Enzymatic Viscosity Reduction and Hydrolysis of Wheat Arabinoxylan in an Industrial Ethanol Fermentation Residue Biotechnol. Prog. 2006, 22, 505-513;
7 Eniko Varga, Helene B. Klinke, Kati Reczey, Anne Belinda Thomsen: High Solid Simultaneous Saccharification and Fermentation of Wet Oxidized Corn Stover to Ethanol BIOTECHNOLOGY AND BIOENGINEERING, VOL. 88, NO. 5, Dec. 5, 2004;
8 WO 2006/56838
9 WO 2007/009463
10 WO8002458
11 US20070274151
12 WO07089144
13 WO07083998
14 US20050105390
15 US20050094486
16 US20030169639

1. A method of processing lignocellulosic biomass comprising;
providing a lignocellulosic biomass
adding an amount of water or aqueous solution sufficient to provide moisture levels between 80-120% of the inherent water holding capacity of the biomass, and
mixing in a mixer that imparts a combination of shear and/or pressing forces such that the biomass is mixed thoroughly within 60 minutes
wherein said mixed biomass having fixed dry matter content is subsequently subject to heat pre-treatment and/or enzymatic hydrolysis.

2. A method of processing lignocellulosic biomass comprising;
providing a lignocellulosic biomass
an amount of water or aqueous solution sufficient to provide moisture levels between 80-120% of the inherent water holding capacity of the biomass, and
mixing thoroughly in a mixer that massages water content into lignocellulosic fibers wherein said mixed biomass having fixed dry matter content is subsequently subject to heat pre-treatment and/or enzymatic hydrolysis.

3. The method of claim 1 wherein the biomass is mixed thoroughly within 30 minutes.

4. The method of claim 1 wherein the biomass is mixed thoroughly within 20 minutes.

5. The method of claim 1 wherein the biomass is mixed thoroughly within 10 minutes.

6. The method of claim 1 or 2 wherein the biomass comprises at least 100 kg.

7. The method of claim 1 or 2 wherein the biomass is characterized by having a distribution of particle sizes prior to pre-treatment having 80% falling within the range of 1 to 10 cm.

8. The method of claim 1 or 2 wherein aqueous solution comprises acids, bases, salts or other chemical additives.

9. The method of claim 1 or 2 wherein water or aqueous solution is added within a vertical column through which biomass is falling.

10. The method of claim 1 or 2 wherein the biomass does not require heat pre-treatment prior to enzymatic hydrolysis.

11. The method of claim 1 or 2 wherein water or aqueous solution has pH between 3.5 and 4.0.

12. The method of claim 1 or 2 wherein water or aqueous solution is added as cold liquid.

13. The method of claim 1 or 2 wherein water or aqueous solution is added as steam or a mixture of steam and liquid.

14. The method of claim 1 or 2 wherein biomass is thoroughly mixed using a twin auger mixer.

15. The method of claim 1 or 2 wherein the biomass comprises one or more of corn stover, wheat straw, rice straw, bagasse, corn fiber, hardwood bulk, softwood bulk, nut shells, corn cobs, grasses, paper, sorted refuse, cotton seed hairs, or empty fruit baskets.
